(19) United States
(12) Patent Application Publication
(10) Pub. No.: US 2004/0005676 A1
(43) Pub. Date: Jan. 8, 2004

(54) Biocatalytic reduction of nitro groups

(75) Inventors: William Greenberg, San Diego, CA (US); David Paul Weiner, Del Mar, CA (US); Brian Adger, Cambridge (GB); Mark J. Burk, San Diego, CA (US)

Correspondence Address: FISH & RICHARDSON, PC, 12390 EL CAMINO REAL, SAN DIEGO, CA, 92130-2081, US

(73) Assignee: Diversa Corporation, San Diego, CA

(21) Appl. No.: 10/351,892

(22) Filed: Jan. 23, 2003

Related U.S. Application Data
(60) Provisional application No. 60/351,624, filed on Jan. 23, 2002.

(51) Int. Cl. C12P 13/00; C12N 9/06; C07K 16/00

(52) U.S. Cl. 435/128; 435/188.5; 435/191

(57) ABSTRACT

The invention provides methods of discovering nitroreductases and biocatalytic methods for reducing compounds comprising a nitro group.

### RELATED APPLICATIONS

This application claims the benefit of priority under 35 U.S.C. §119(e) of U.S. Provisional Application No. 60/351,624, filed Jan. 23, 2002. The aforementioned application is explicitly incorporated herein by reference in its entirety and for all purposes.

### TECHNICAL FIELD

This invention relates to the field of pharmaceuticals and synthetic chemistry, and more particularly to methods of discovering nitroreductases and biocatalytic methods for reducing compounds comprising a nitro group.

### BACKGROUND

Nitroreductases (NRs) can catalyze the six-electron reduction of nitro compounds to the corresponding amines. Amines have a variety of applications as synthons and advanced pharmaceutical intermediates. There are markets for both aromatic amines and chiral aliphatic amines.

There are two main classes of nitroreductases that have been described from a number of microorganisms. These are the oxygen-sensitive and oxygen-insensitive nitroreductases. The oxygen-sensitive enzyme can catalyze nitroreduction only under anaerobic conditions. A nitro anion radical is formed by a one-electron transfer and is immediately reoxidized in the presence of oxygen thus generating a futile cycle whereby reducing equivalents are consumed without nitroreduction. On the other hand the oxygen-insensitive nitroreductases catalyze nitroreduction in a series of two electron transfers, first via the nitroso and then the hydroxylamine intermediates before forming the amine. To date, a limited number of aromatic nitro substrates for nitroreductases have been examined with a focus on bioremediation and prodrug activation.

Many anilines when prepared from nitrobenzenes by reduction with Pd catalysts can give explosive intermediates. This has caused problems on scale. The Pd catalyzed amination of aromatic halides is an alternative entry into aromatic amines. However this is limited in substrate scope.

### SUMMARY

The invention provides biocatalytic applications of NR enzymes for the synthesis of value-added amines.

The invention provides biocatalytic methods for reducing a compound comprising a nitro group, the method comprising the following steps: (a) providing a nitroreductase; (b) providing a compound comprising a nitro group; and (c) contacting the nitroreductase with the compound under conditions wherein the nitro group is reduced to an amine group. In one aspect, the contacting is performed in a reaction vessel, a cell extract or a whole cell. The cell can be a bacterial cell, a yeast cell, a fungal cell, a plant cell, an insect cell or a mammalian cell.

The nitroreductase can be a recombinant, isolated or synthetically generated enzyme. The nitroreductase can be a recombinant, isolated or synthetically generated biocatalytic antibody having a nitroreductase activity.

In one aspect, the compound comprises a nitroaromatic compound and a substituted aniline is biocatalytically generated. The nitroaromatic compound can be halosubstituted. The compound can comprise a nitroalkyl group. The compound can comprise a nitroalkane or a nitroaromatic, such as a 2-nitro-benzoic acid. In one aspect, the nitroreductase reduces 2-nitro-benzoic acid to anthranilic acid.

In one aspect, the compound comprises a styrene. The compound can comprise a racemic nitroalkane compound and a chiral amine is biocatalytically generated. In one aspect, compound can comprises a 4-nitrobuylamine or a 1,4-dinitro-butane. In one aspect, a putrescine is biocatalytically generated.

The invention provides methods for selecting a nitroreductase that can catalyze the reduction of a nitro group to its corresponding amine comprising the following steps: (a) providing a test sample comprising a polypeptide; (b) providing a compound comprising a nitro group; and (c) contacting the test sample with the compound comprising a nitro group and detecting the generation of a corresponding amine, wherein the generation of the corresponding amine indicates the presence of a nitroreductase in the test sample.

The invention provides methods for selecting a nitroreductase that can catalyze the reduction of an aliphatic nitro group comprising the following steps: (a) providing a test sample comprising a polypeptide; (b) providing a 4-nitrobuylamine or a 1,4-dinitro-butane; and (c) contacting the test sample with the 4-nitrobuylamine or the 1,4-dinitro-butane and detecting the generation of putrescine, wherein the generation of putrescine indicates the presence of a nitroreductase in the test sample. In one aspect, the method for selecting a nitroreductase that can catalyze the reduction of an aliphatic nitro group comprises the following steps: (a) providing a test sample comprising a polypeptide; (b) providing a 4-nitrobuylamine or a 1,4-dinitro-butane; and (c) contacting the test sample with the 4-nitrobuylamine or the 1,4-dinitro-butane in an in vivo system or equivalent system and detecting the generation of succinate, wherein the generation of succinate indicates the presence of a nitroreductase in the test sample. In one aspect, the putrescine or the succinate is detected by on-line HPLC or by using a mass spectograph.

The invention provides methods for selecting a nitroreductase that can catalyze the reduction of an aliphatic nitro group comprising the following steps: (a) providing a test sample comprising a polypeptide; (b) providing a 2-nitro-benzoic acid; and (c) contacting the test sample with the 2-nitro-benzoic acid system and detecting the generation of anthranilic acid, wherein the generation of anthranilic acid indicates the presence of a nitroreductase in the test sample. In one aspect, the anthranilic acid is detected by fluorescence, on-line HPLC or by using a mass spectograph.

The invention provides methods for selecting a nitroreductase that can catalyze the reduction of an aliphatic nitro group comprising the following steps: (a) providing a test sample comprising a polypeptide; (b) providing a 2-nitro-benzoic acid; and (c) contacting the test sample with the 2-nitro-benzoic acid and detecting the generation of anthranilic acid, wherein the generation of anthranilic acid indicates the presence of a nitroreductase in the test sample.

The invention provides methods for selecting a nitroreductase that can catalyze the reduction of an aliphatic nitro group comprising the following steps: (a) providing a test sample comprising a polypeptide; (b) providing a 2-nitro-benzoic acid; and (c) contacting the test sample with the 2-nitro-benzoic acid in an in vivo system or equivalent system and detecting the generation of tryptophan, wherein the generation of tryptophan indicates the presence of a nitroreductase in the test sample.

The invention provides methods for selecting a nitroreductase that can catalyze the reduction of an aliphatic nitro group comprising the following steps: (a) providing a test sample comprising a polypeptide; (b) providing a nitroacid; and (c) contacting the test sample with the nitroacid and detecting the generation of an amino acid, wherein the generation of the amino acid indicates the presence of a nitroreductase in the test sample. The test sample and the nitroacid can be contacted in an in vivo system. The in vivo system can comprise an amino acid auxotroph.

The invention provides methods for selecting a nitroreductase that can catalyze the reduction of a halogenated nitroaromatic compound comprising the following steps: (a) providing a test sample comprising a polypeptide; (b) providing a halogenated nitroaromatic compound; and (c) contacting the test sample with the halogenated nitroaromatic compound and detecting the generation of a corresponding amine, wherein the generation of the corresponding amine indicates the presence of a nitroreductase in the test sample.

The invention provides methods for selecting a nitroreductase that can catalyze the reduction of a styrene comprising the following steps: (a) providing a test sample comprising a polypeptide; (b) providing a styrene; and (c) contacting the test sample with the styrene and detecting the generation of a corresponding amine, wherein the generation of the corresponding amine indicates the presence of a nitroreductase in the test sample.

The invention provides methods for selecting a nitroreductase that can catalyze the reduction of an aliphatic nitrocompound comprising the following steps: (a) providing a test sample comprising a polypeptide; (b) providing an aliphatic nitrocompound; and (c) contacting the test sample with the aliphatic nitrocompound and detecting the generation of a corresponding amine, wherein the generation of the corresponding amine indicates the presence of a nitroreductase in the test sample. In one aspect, the corresponding amine is a chiral amine.

The invention provides methods for selecting a nitroreductase that can catalyze the reduction of an asymmetric nitroaldol comprising the following steps: (a) providing a test sample comprising a polypeptide; (b) providing an asymmetric nitroaldol; (c) contacting the test sample with the asymmetric nitroaldol and detecting the generation of a corresponding amine, wherein the generation of the corresponding amine indicates the presence of a nitroreductase in the test sample.

The invention provides methods for chemoselective reduction of a nitro group, the method comprising the following steps: (a) providing a nitroreductase; (b) providing a nitro group; and (c) contacting the nitroreductase with the nitro group under conditions wherein the nitroreductase catalyzes the reduction of the nitro group to an amine group.

The invention provides biocatalytic methods for making a substituted aniline, the method comprising the following steps: (a) providing a nitroreductase; (b) providing a nitroaromatic compound; and (c) contacting the nitroreductase with the nitroaromatic compound under conditions wherein the nitroreductase catalyzes the reduction of the nitro group to an amine group and a substituted aniline is generated. The nitroaromatic compound can comprise a halogenated nitroaromatic compound.

The invention provides biocatalytic methods for reducing a styrene, the method comprising the following steps: (a) providing a nitroreductase; (b) providing a styrene; and (c) contacting the nitroreductase with the styrene under conditions wherein the nitroreductase catalyzes the reduction of the nitro group to an amine group and the styrene is reduced to its corresponding amine.

The invention provides biocatalytic methods for making a chiral amine, the method comprising the following steps: (a) providing a nitroreductase; (b) providing a racemic nitroalkane compound; and (c) contacting the nitroreductase with the nitroalkane under conditions wherein the nitroreductases reduces the nitroalkane to generate a chiral amine. The contacting can take place at a neutral pH such that a dynamic kinetic resolution is effected. The nitroalkane compound can comprise a benzylic nitro group.

The invention provides biocatalytic methods for making putrescine, the method comprising the following steps: (a) providing a nitroreductase; (b) providing a 4-nitrobuylamine or a 1,4-dinitro-butane; and (c) contacting the nitroreductase with the 4-nitrobuylamine or 1,4-dinitro-butane under conditions wherein the nitroreductases reduces the 4-nitrobuylamine or 1,4-dinitro-butane to generate a putrescine.

The invention provides methods for selecting a nucleic acid encoding a nitroreductase, the method comprising the following steps: (a) providing a plurality of nucleic acids; (b) expressing the nucleic acids in a system comprising a nitro compound; and (c) detecting the system where the nitro compound has been reduced to its corresponding amine, thereby selecting a nucleic acid encoding a nitroreductase. The plurality of nucleic acids can comprise a genomic library or a cDNA library. In one aspect, the plurality of nucleic acids comprises an environmental library. In one aspect, the system lacks or has insubstantial amounts of an endogenous nitroreductase. In one aspect, the plurality of nucleic acids are cloned in an expression cassette, such as a phage, a phagemid, a plasmid or a recombinant virus. In one aspect, the system comprises an in vitro system. In one aspect, the system comprises a cell, such as a bacterial cell.

In one aspect, the corresponding amine is a cell growth factor or a compound the cell lacks in sufficient amount that is necessary for cell growth or survival, and expression of a nitroreductase reduces the nitro compound to its corresponding amine such that only cells expressing a nitroreductase proliferate or survive, thereby allowing selection of a nucleic acid encoding a nitroreductase. The nitro compound can comprise a 4-nitrobuyl amine or a 1,4-dinitro-butane and the corresponding amine growth factor is putrescine.

In one aspect, the corresponding amine is a precursor or an intermediate in the synthesis of a cell growth factor or a compound that is necessary for cell growth or survival that the cell lacks in sufficient amounts, and expression of a nitroreductase reduces the nitro compound to its corresponding amine such that only cells expressing a nitroreductase proliferate or survive, thereby allowing selection of a nucleic acid encoding a nitroreductase. The nitro compound can comprise a 4-nitrobuylamine or a 1 ,4-dinitro-butane and the corresponding amine that the cell lacks in sufficient amount that is necessary for cell growth is succinate, expression of a nitroreductase generates sufficient succinate for the cell to proliferate or survive.

In one aspect, the cell lacks or substantially lacks anthranilate synthase activity and the ability to generate tryptophan, the nitro compound comprises a 2-nitro-benzoic acid, and expression of a nitroreductase generates sufficient tryptophan for the cell to proliferate or survive.

In one aspect, the nitro compound comprises a nitro acid and the corresponding amine that the cell lacks in sufficient amount that is necessary for cell growth is an amino acid, and expression of a nitroreductase generates sufficient amino acid for the cell to proliferate or survive. In one aspect, the cell is an amino acid auxotroph, such as a leucine auxotroph, a proline auxotroph or a tryptophan auxotroph.

The invention provides methods for making a nucleic acid encoding a nitroreductase capable of catalyzing reduction of a nitro compound in a set of conditions, the method comprising the following steps: (a) providing a nucleic acid encoding a first nitroreductase; (b) modifying the nitroreductase-encoding nucleic acid to generate a plurality of modified nucleic acids; (c) expressing the modified nucleic acids in a system comprising a nitro compound in the set of conditions and detecting if the nitro compound has been reduced to its corresponding amine; (d) detecting which modified nucleic acid encoded the nitroreductases that catalyzed the reduction of the nitro compound to its corresponding amine, thereby making a nucleic acid encoding a modified nitroreductase capable of catalyzing reduction of a nitro compound in a set of conditions.

The invention provides methods for making a nitroreductase capable of catalyzing reduction of a nitro compound in a set of conditions, the method comprising the following steps: (a) providing a nucleic acid encoding a first nitroreductase; (b) modifying the nitroreductase-encoding nucleic acid to generate a plurality of modified nucleic acids; (c) expressing the modified nucleic acids in a system comprising a nitro compound in the set of conditions and detecting if the nitro compound has been reduced to its corresponding amine; and, (d) detecting which nitroreductase catalyzed the reduction of the nitro compound to its corresponding amine, thereby making a modified nitroreductase capable of catalyzing reduction of a nitro compound in a set of conditions. In one aspect, the first nitroreductase is not active in the selected set of conditions. In one aspect, the conditions in which the modified nitroreductase has optimal activity differs from the conditions in which the first nitroreductase has optimal activity. In one aspect, the modified nitroreductase acts on a different substrate than the first nitroreductase. In one aspect, the modified nitroreductase produces a different product than the first nitroreductase. In one aspect, the modified nitroreductases has a different co-factor requirement that the first nitroreductase. In one aspect, the set of conditions comprise high or low temperatures, high or low pH or high or low salt conditions.

In one aspect, the nucleic acids expressing the modified nitroreductases are cloned in expression vehicles, such as a phage, a phagemid or an expression vector.

In one aspect, the modified nucleic acids or modified nitroreductases are expressed in a well in a microtiter plate. In one aspect, the modified nucleic acids or modified nitroreductases are expressed in a capillary tube, such as a capillary array, e.g., a GIGAMATRIX™.

In one aspect, a modified nucleic acid sequence is generated by a method comprising gene site saturated mutagenesis (GSSM). In one aspect, a modified nucleic acid sequence is generated by a method selected from the group consisting of gene site saturated mutagenesis (GSSM), error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, synthetic ligation reassembly (SLR) and a combination thereof In one aspect, a modified nucleic acid sequence is generated by a method selected from the group consisting of recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

In one aspect, the method is repeated iteratively to generate a modified nitroreductase having a desired activity under a particular set of conditions, a modified nitroreductase using a desired substrate or a modified nitroreductase generating a desired product.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is an illustration of an exemplary method of the invention, as described in detail below.

FIG. 2 is an illustration of an exemplary method of the invention, as described in detail below.

FIG. 3 schematically illustrates how 4-nitrobutylamine and 1,4-dinitrobutane can act as substrates for nitroreductases (NR), and how products of that reaction can then be converted to succinate, as described in detail below.

FIG. 4 schematically illustrates how 2-nitro-benzoic acid can be used as a substrate for nitroreductase to generate anthranilic acid, as described in detail below.

FIG. 5 schematically illustrates how nitroacids can be used as a substrate for nitroreductase to generate amino acids, as described in detail below

FIG. 6A schematically illustrates the reduction of a nitroaromatic compound to form a substituted aniline, as described in detail below. FIG. 6B schematically illustrates the reduction of a nitroalkane compound to form a chiral amine, as described in detail below.

FIG. 7 illustrates exemplary methods comprising the biocatalytic conversion of halogenated nitroaromatic compounds to their corresponding amines and the biocatalytic conversion of styrenes to their corresponding amines, as described in detail below.

FIG. 8A illustrates exemplary methods comprising biocatalytic asymmetric Henry Reactions and FIG. 8B illustrates biocatalytic asymmetric Michael Reactions, as described in detail below

Like reference symbols in the various drawings indicate like elements.

### DETAILED DESCRIPTION

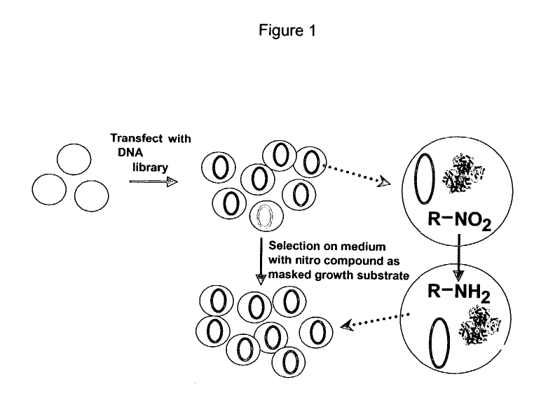

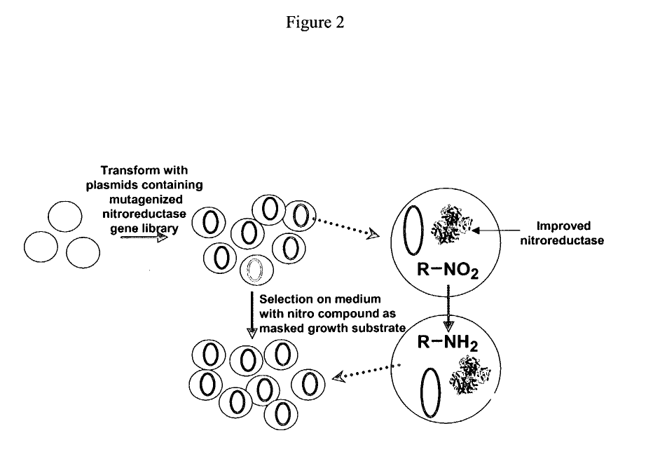

The invention provides methods for the discovery or evolution of a wide range of nitroreductases, exemplary aspects of which are illustrated in FIGS. 1 and 2. In one aspect, the methods of the invention are used as ultra-high throughput methodologies for the discovery of nitroreductases. In one aspect, a selection strategy of the methods of the invention comprises inserting into a system a nucleic acid or a plurality of nucleic acids, such as a DNA library (e.g., from an environmental library), and determining whether a nitro compound is converted (reduced) to its corresponding amine. The system can be an in vitro system or an in vivo system, such as a cell. In one aspect, the generation of the corresponding amine is determined by introducing a nucleic acid into an in vitro translation system or an in vivo system, such as a cell, or, introducing a sample of the system (e.g., comprising a polypeptide expressed by a nucleic acid, e.g., a nucleic acid of a library) into an in vitro system or an in vivo system, such as a cell, and then testing the system (e.g., the cell) for its ability to convert a nitro compound into its corresponding amine. The amine can be a necessary growth factor or the amine can produce a detectable signal or act directly or indirectly to produce a detectable signal.

In one aspect, instead of inserting a nucleic acid or a plurality of nucleic acids into a system (an in vitro system or an in vivo system), nucleic acids already in a system are modified either stochastically or non-stochastically. For example, the genetic makeup of a cell can be stochastically or non-stochastically altered and then the cell is tested for its ability to convert a nitro compound into its corresponding amine, wherein the amine is a necessary growth factor or the amine produces a detectable signal or acts directly or indirectly to produce a detectable signal. Alternatively, the genetic makeup of an in vitro system comprising a library can be stochastically or non-stochastically altered and samples expression products of that system can be tested for production of an amine compound.

In one aspect, a plurality of nucleic acids, e.g., in the form of a library, e.g., representing the DNA repertoire of a library, such as an environmental library, is inserted into expression vehicles, e.g., vectors, plasmids, recombinant viruses, phages, phagemids and the like, and then these constructs are inserted into a cell or an in vitro expression system. Then the expression vehicle-comprising cells (e.g., vector-comprising cells, phage or phagemid-containing cells) are grown in medium lacking a crucial growth factor, wherein that growth factor is a compound comprising an amine. In one aspect, the cells are provided the nitro compound that is the precursor of the amine, wherein the nitro compound can be converted to the amine-comprising growth factor by a nitroreductase enzyme. It is then determined which cells divide, divide faster or slower and/or do not die. Alternatively, the amine can be directly detected (e.g., by HPLC, mass spec and the like) or indirectly detected by its ability to produce a detectable signal or act directly or indirectly to produce a detectable signal.

In one aspect, the expression vehicle-comprising cells (e.g., vector-comprising cells, phage or phagemid-containing cells) are grown in medium having a nitroreductase substrate, wherein a nitroreductase can convert the substrate into a product that can be directly or indirectly measured (monitored). The substrate can be added to the cell or can be endogenous to the cell. Alternatively, the expression vehicles can be expressed in an in vitro system (e.g., cell-free in vitro translation system) comprising a nitroreductase substrate, wherein a nitroreductase can convert the substrate into a product that can be directly or indirectly measured (monitored).

The steps of an exemplary method are summarized below:

A) Phagemid library-comprising cells are inoculated into media lacking a crucial amine growth source (the cells cannot themselves synthesize the crucial amine growth source). In alternative aspects, both solid and liquid media and/or selection systems are used.

B) A nitro compound that is a direct precursor to the amine growth source is added to the medium.

C) Only cells generating an active nitroreductase enzyme (pink/red-colored phagemid in FIG. 1) capable of reducing the nitro compound to the amine growth substrate will grow and proliferate.

The selection methodologies of the invention are also applicable to the “directed evolution” of a nitroreductase in order to change (e.g., “improve”) its catalytic or other properties. In one aspect, conditions of the selection are adjusted such that clones expressing wild type nitroreductases are unable to grow but clones containing modified or “improved” nitroreductases can grow, or grow faster, or otherwise distinguish themselves from cells not having an nitroreductase that has activity under a desired condition. The “desired condition” can be high or low temperature, high or low pH, high or low salt conditions, aerobic or anaerobic conditions, and the like.

Nucleic acids encoding nitroreductase can be “modified” by any methodology, including stochastic or non-stochastic protocols, or a combination thereof. “Modified” nucleic acid encoding “modified” nitroreductases can be cloned into an expression vehicle, e.g., anything from an expression cassette (simply comprising a promoter and a coding sequence) to any expression vehicle, e.g., a vector such as a phage, phagemid, plasmid, recombinant virus and the like. In one aspect, libraries of plasmid-borne mutagenized genes are generated from gene saturation mutagenesis (e.g., GSSM), gene reassembly, or other mutagenesis technique, or combination thereof. Nucleic acids encoding an enzyme, e.g., a nitroreductase, can be “modified” by, e.g., gene site saturated mutagenesis (GSSM), stepwise nucleic acid reassembly, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, synthetic ligation reassembly (SLR) or a combination thereof. Nucleic acids encoding an enzyme, e.g., a nitroreductase, can be modified by, e.g., recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation or a combination thereof. In one aspect, a nucleic acid encoding a nitroreductase selected by the method of the invention is modified and re-screened for its ability to reduce a compound comprising a nitro group to an amine. This process can be iteratively repeated, and the second (or further) screening(s) can be done under different conditions from the first (or earlier) screening(s).

In one aspect, a nucleic acid library (e.g., an environmental library) is transformed into a suitable host and then subjected to a selection process, e.g., a growth selection or any selection system that can determine whether a nitroreductase has been expressed, e.g., whether a nitroreductase has been generated that can catalyze the reduction of a nitro compound to an amine. Only clones expressing a nitroreductase under the desired conditions (e.g., a “more active” nitroreductase) will be detected, e.g., only those clones will grow and proliferate. These can then be subjected to further iterative rounds of mutagenesis (nucleic acid sequence modification) and selection.

Any nitro compound that can be reduced to an amine can be used to practice the methods of the invention. For example, any nitro compound can be used to select for the presence of an active nitroreductases (by the enzyme's reduction of the nitro compound to an amine). The amine generated by the enzyme's catalytic activity can be directly or indirectly detected. For example, the amine can be directly detected by an antibody or by an on-line detection system, e.g., an HPLC, mass spectrograph, a chromatographic detection system or the like. Alternatively, the amine can be indirectly detected, e.g., by its ability to stimulate the growth of the cell. The amine can also be indirectly detected by its ability to act as a substrate in a chemical reaction, a product of which is detected, e.g., by colorimetric or other means. In one aspect, the amine is detected by its acting as a growth source in an appropriate host cell. The amine can be an essential growth factor for the cell or can be a factor that detectably decreases or increases the rate of growth of a cell or allows the cell to grow under modified conditions.

The following are exemplary selection substrates used in the methods of the invention; however, the methods are not limited to just these examples.

Putrescine (1,4-diaminobutane)

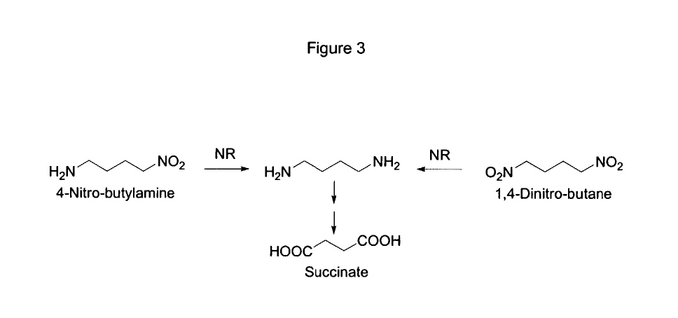

In one aspect, 4-nitrobuylamine or 1,4-dinitro-butane (see FIG. 3) are used as nitroreductases selection substrates. Nitroreductases that can catalyze the reduction of aliphatic nitro groups can be selected for using either 4-nitrobuylamine and/or 1,4-dinitrobutane. Both of these substrates can be reduced to putrescine (1,4-diaminobutane). The putrescine can be detected directly (e.g., by on-line HPLC or mass spec) or indirectly. For example, the putrescine can be converted via a series of enzymatic steps into succinate. The succinate or any of the generated intermediates can be detected directly or indirectly. For example, the succinate can then enter the TCA cycle. In one aspect, a cell, such as a bacterial cell, can utilize putrescine as a growth source, e.g., an essential growth source. In one aspect, an E. coli that can utilize putrescine as a growth source is used (e.g., E. coli SEL 700).

Anthanilate

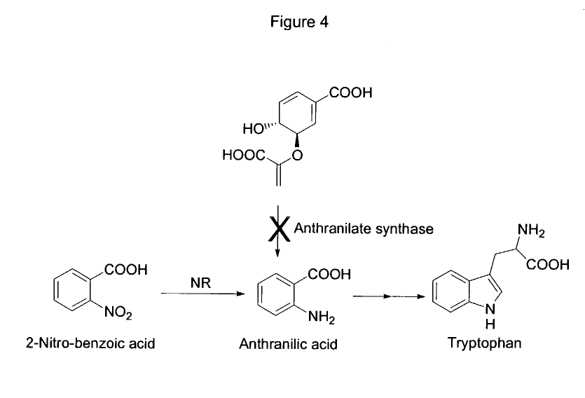

In one aspect, nitroreductases with specificities for nitroaromatics are selected for using 2-nitro-benzoic acid, as illustrated in FIG. 4. In one aspect, the nitroreductase reduces 2-nitro-benzoic acid to anthranilic acid. The anthranilic acid can be detected directly (e.g., by on-line HPLC or mass spec) or indirectly. For example, anthranilic acid is fluorescent; thus, the conversion of nitrobenzoic acid to anthranilic acid can be the basis of a fluorogenic screen, e.g., by a capillary array system, such as a GIGAMATRIX™ (Diversa Corporation, San Diego, Calif.) screening technology.

In one aspect, the anthranilic acid can be converted via a series of enzymatic steps into tryptophan. The tryptophan or any of the generated intermediates can be detected directly (e.g., by on-line HPLC or mass spec) or indirectly. For example, the tryptophan can be used in tryptophan biosynthesis. In one aspect, a selection strain lacking or substantially lacking anthranilate synthase activity (e.g., an anthranilate synthase knockout) is used to prevent tryptophan biosynthesis from chorismate (see FIG. 4).

Amino Acid Precursors

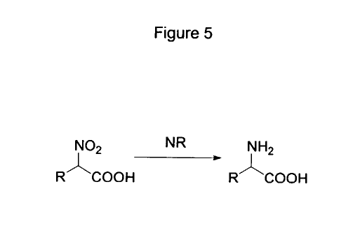

In one aspect, nitroacids that can be reduced to amino acids by nitroreductases are used are substrates in the methods of the invention (see FIG. 5). Nitroacids can be used to select for nitroreductases capable of forming amino acids. The amino acids can be detected directly (e.g., by on-line HPLC or mass spec) or indirectly, e.g., where the amino acid is an essential growth factor. For example, amino acid auxotrophic host strains can be used, e.g., strains including but not limited to leucine and proline auxotrophs.

Novel Routes to Amines Using Nitroreductases

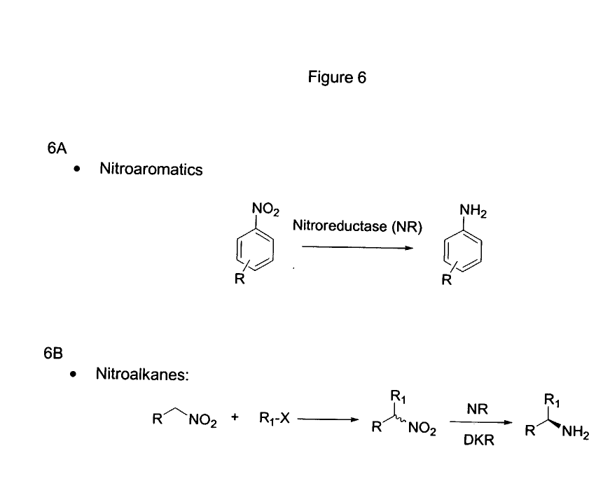

The invention provides several novel biocatalytic applications of nitroreductases. In some aspects, these new biocatalytic applications enable an efficient cost-effective synthesis for a variety of value-added amines, for example substituted anilines and chiral amines, as illustrated in FIGS. 6A and 6B, respectively. In these aspects of the invention, any nitroreductase enzyme, or equivalent, e.g., a catalytic antibody, can be used, including isolated, recombinant or synthetic forms.

Substituted Anilines from Nitroaromatics

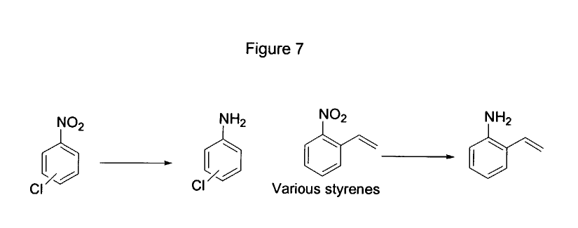

In one aspect, the invention provides methods of making substituted anilines using a nitroreductase enzyme, or equivalent, e.g., a catalytic antibody. In this aspect, a nitroreductase enzyme, or equivalent, catalyzes the reduction of a nitroaromatic compound to form a substituted aniline, as illustrated in FIG. 6A and FIG. 7. FIG. 7 illustrates exemplary methods comprising the biocatalytic conversion of halogenated nitroaromatic compounds to their corresponding amines and the biocatalytic conversion of styrenes to their corresponding amines.

The invention also provides a biocatalytic route using nitroreductases catalyzing the opposite reaction, i.e., for the preparation of nitroaromatic compounds from substituted anilines. In one aspect, the biocatalytic route of the invention is a mild method compatible with substituted anilines that would not tolerate Pd/C reduction or the Buchwald/Hartwig chemistry.

Chiral Amines from Nitroalkanes

In one aspect, the invention provides methods of making chiral amines using a nitroreductase enzyme, or equivalent, e.g., a catalytic antibody. In this aspect, a nitroreductase enzyme, or equivalent, catalyzes the reduction of an aliphatic nitrocompound (e.g., a nitroalkane compound) to form a chiral amine, as illustrated in FIG. 6B. Base-catalyzed racemization allows dynamic kinetic resolution of the product.

In one aspect, the method further comprises use of dynamic kinetic resolution (DKR, see, e.g., Kim (2002) Curr. Opin. Biotechnol. 13:578-587). In one aspect, the acidity of the position a to the nitro group enables racemization to occur at neutral pHs such that a dynamic kinetic resolution is effected. In one aspect, a 100% theoretical yield of chiral amine is possible starting from a racemic nitro compound. In the cases of a benzylic nitro compound the racemization is further facilitated.

The invention also provides a biocatalytic route using nitroreductases catalyzing the opposite reaction, i.e., for the preparation of nitroalkanes from chiral amines.

Other Biocatalytic Reactions

Any biocatalytic reaction reducing a nitro compound to the corresponding amine using a nitroreductase or equivalent is within the scope of the invention. As noted above, any nitroreductase enzyme, or equivalent, e.g., a catalytic antibody, can be used, including isolated, recombinant or synthetic forms.

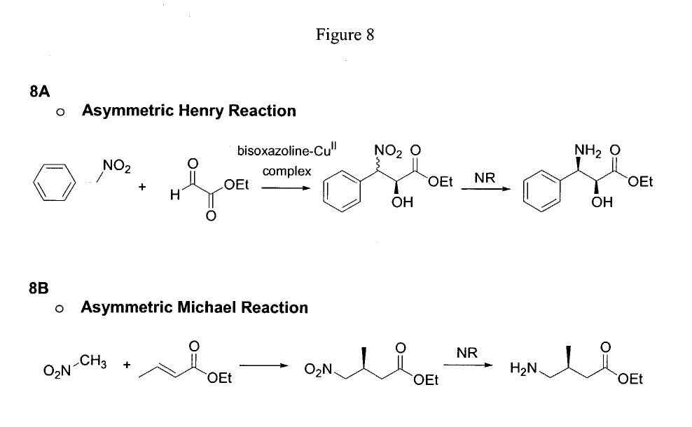

Another exemplary biocatalytic reaction of the invention comprises an asymmetric Henry Reaction (an asymmetric nitroaldol (Henry) reaction, see, e.g., Trost (2002) Org. Lett. 4:2621-2623; Davis (2001) Org. Lett. 3:3075-3078; Ballini (1997) J. Org. Chem. 62:425-427), as set forth in FIG. 8A. The invention also provides a biocatalytic route using nitroreductases catalyzing the opposite reaction.

Another exemplary biocatalytic reaction of the invention comprises an asymmetric Michael Reaction (Michael addition reactions, see, e.g., Cave (2001) Chem. Commun. (Camb) 21:2159-2169, U.S. Pat. No. 5,350,875), as set forth in FIG. 8B. The Michael reaction is a known process wherein a Michael acceptor (such as an alpha, beta-ethylenically-unsaturated aldehyde, ester, nitrile, ketone, sulfone, or sulfoxide) is reacted with a Michael donor (such as a dialkyl malonate) to elongate a carbon chain. The invention also provides a biocatalytic route using nitroreductases catalyzing the opposite reaction.

Process Considerations

Nitroreductases are flavin-dependent enzymes that catalyze reduction of nitro compounds using NADPH or NADH and flavin mononucleotides. In alternative aspects, the methods use systems comprising flavin mononucleotides (FMN and/or FAD) and NADH and/or NADPH co-factors. In one aspect, these cofactors and other equivalent or, as appropriate, necessary cofactors are added to the system. In alternative aspects, a whole cell process or cofactor regeneration systems (in vivo and in vitro) are used. Electrochemical approaches may also be utilized. In whole cell applications, mass transfer issues may be important. The redox potentials available from flavin mononucleotides (FMN) in the nitroreductase active site environment may be crucial in determining whether aliphatic nitro compounds can be reduced.

Generating and Manipulating Nucleic Acids

The invention provides methods of making a nitroreductase capable of catalyzing reduction of a nitro compound in a particular, or desired, set of conditions comprising the modification and expression of nitroreductases-encoding nucleic acids. The invention also provides methods for selecting a nucleic acid encoding a nitroreductase. In practicing the methods of the invention, the nucleic acid can be cloned into and expressed using expression vehicles of any kind, e.g., expression cassettes such as expression vectors, encoding nitroreductases modified by the methods of the invention. The invention also includes methods for discovering new nitroreductase sequences using modified, or mutated, nucleic acids. In practicing the methods of the invention, nucleic acids can be modified by any technique, e.g., by synthetic ligation reassembly, optimized directed evolution system or gene saturation mutagenesis, such as GSSM.

The phrases “nucleic acid” or “nucleic acid sequence” can include an oligonucleotide, nucleotide, polynucleotide, or to a fragment of any of these, to DNA or RNA (e.g., mRNA, rRNA, tRNA) of genomic or synthetic origin which may be single-stranded or double-stranded and may represent a sense or antisense strand, to peptide nucleic acid (PNA), or to any DNA-like or RNA-like material, natural or synthetic in origin, including, e.g., iRNA, ribonucleoproteins (e.g., iRNPs). The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156.

The nucleic acids used to practice the invention the invention can be made, isolated and/or manipulated by, e.g., cloning and expression of CDNA libraries, amplification of message or genomic DNA by PCR, and the like. The invention can be practiced in conjunction with any method or protocol or device known in the art, which are well described in the scientific and patent literature.

General Techniques

The nucleic acids used to practice the methods of the invention include RNA, CDNA, iRNA, antisense nucleic acid, cDNA, genomic DNA, vectors, viruses or hybrids thereof, that can be isolated from a variety of sources, genetically engineered, amplified, and/or expressed/ generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440□03444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In one aspect, a nucleic acid encoding a polypeptide is assembled in appropriate phase with a leader sequence capable of directing secretion of the translated polypeptide or fragment thereof.

Transcriptional and Translational Control Sequences

In practicing the methods of the invention, in some aspects nucleic acid (e.g., DNA) sequences are operatively linked to expression (e.g., transcriptional or translational) control sequence(s), e.g., promoters or enhancers, to direct or modulate RNA synthesis/ expression. The expression control sequence can be in an expression vector. Exemplary bacterial promoters include lacI, lacZ, T3, T7, gpt, lambda PR, PL and trp. Exemplary eukaryotic promoters include CMV immediate early, HSV thymidine kinase, early and late SV40, LTRs from retrovirus, and mouse metallothionein I.

Promoters suitable for expressing a polypeptide in bacteria include the E. coli lac or trp promoters, the lacI promoter, the lacZ promoter, the T3 promoter, the T7 promoter, the gpt promoter, the lambda PR promoter, the lambda PL promoter, promoters from operons encoding glycolytic enzymes such as 3-phosphoglycerate kinase (PGK), and the acid phosphatase promoter. Eukaryotic promoters include the CMV immediate early promoter, the HSV thymidine kinase promoter, heat shock promoters, the early and late SV40 promoter, LTRs from retroviruses, and the mouse metallothionein-I promoter. Other promoters known to control expression of genes in prokaryotic or eukaryotic cells or their viruses may also be used.

Expression Vectors and Cloning Vehicles

In practicing the methods of the invention, in some aspects nucleic acids are cloned into expression vectors and cloning vehicles. Expression vectors and cloning vehicles can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors used to practice the methods of the invention can include chromosomal, non-chromosomal and synthetic DNA sequences. Large numbers of suitable vectors are known to those of skill in the art, and are commercially available. Exemplary vectors are include: bacterial: pQE vectors (Qiagen), pBluescript plasmids, pNH vectors, (lambda-ZAP vectors (Stratagene); ptrc99a, pKK223-3, pDR540, pRIT2T (Pharmacia); Eukaryotic: pXT1, pSG5 (Stratagene), pSVK3, pBPV, pMSG, pSVLSV40 (Pharmacia). However, any other plasmid or other vector may be used so long as they are replicable and viable in the host. Low copy number or high copy number vectors may be employed with the present invention.

Expression vectors used to practice the methods of the invention may comprise a promoter, a ribosome binding site for translation initiation and a transcription terminator. The vector may also include appropriate sequences for amplifying expression. Mammalian expression vectors can comprise an origin of replication, any necessary ribosome binding sites, a polyadenylation site, splice donor and acceptor sites, transcriptional termination sequences, and 5′ flanking non-transcribed sequences. In some aspects, DNA sequences derived from the SV40 splice and polyadenylation sites may be used to provide the required non-transcribed genetic elements.

In one aspect, expression vectors used to practice the methods of the invention contain one or more selectable marker genes to permit selection of host cells containing the vector. Such selectable markers include genes encoding dihydrofolate reductase or genes conferring neomycin resistance for eukaryotic cell culture, genes conferring tetracycline or ampicillin resistance in E. coli, and the S. cerevisiae TRP1 gene. Promoter regions can be selected from any desired gene using chloramphenicol transferase (CAT) vectors or other vectors with selectable markers.

Vectors used to practice the methods of the invention may also contain enhancers to increase expression levels. Enhancers are cis-acting elements of DNA, usually from about 10 to about 300 bp in length that act on a promoter to increase its transcription. Examples include the SV40 enhancer on the late side of the replication origin bp 100 to 270, the cytomegalovirus early promoter enhancer, the polyoma enhancer on the late side of the replication origin, and the adenovirus enhancers.

The vector used to practice the methods of the invention may be in the form of a plasmid, a viral particle, a phagemid, or a phage. Other vectors include chromosomal, nonchromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook.

Host Cells and Transformed Cells

In one aspect, the invention provides methods for the discovery or evolution of a wide range of nitroreductases by methods comprising introducing a nucleic acid into in vivo system, such as a cell, and then testing the cell for its ability to convert a nitro compound into its corresponding amine. In some aspects, in practicing the methods of the invention transformed cells are used to express and/or screen for modified nitroreductases-encoding nucleic acid sequences. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art. Techniques for transforming a wide variety of higher plant species are well known and described in the technical and scientific literature. See, e.g., Weising (1988) Ann. Rev. Genet. 22:421-477, U.S. Pat. No. 5,750,870.

In some aspects, in practicing the methods of the invention cell-free translation systems can also be employed to produce (express) a polypeptide. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Amplification of Nucleic Acids

In practicing the methods of the invention, modified and unmodified nucleic acids encoding polypeptides (e.g., nitroreductases) can be reproduced by, e.g., amplification. One of skill in the art can design amplification primer sequence pairs for any part of or the full length of a desired sequence. Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified. The skilled artisan can select and design suitable oligonucleotide amplification primers. Amplification methods are also well known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

Modification of Nucleic Acids

The invention provides methods of making modified nitroreductases by generating variants of nitroreductase-encoding nucleic acids. In one aspect, the invention provides methods of making a nitroreductase capable of catalyzing reduction of a nitro compound in a particular, or desired, set of conditions comprising the modification and expression of nitroreductase-encoding nucleic acids. These methods can be repeated or used in various combinations to generate nitroreductases having an altered or different activity or an altered or different stability from that of a starting, or “wild type” nitroreductase encoded by a template nucleic acid. These methods also can be repeated or used in various combinations, e.g., to generate variations in gene/message expression, message translation or message stability. In another aspect, the genetic composition of a cell is altered by, e.g., modification of a homologous gene ex vivo, followed by its reinsertion into the cell.

In practicing the methods of the invention, a nucleic acid (e.g., a nitroreductase-encoding nucleic acid) can be altered by any means. For example, random or stochastic methods, or, non-stochastic, or “directed evolution,” methods, see, e.g., U.S. Pat. No. 6,361,974. Methods for random mutation of genes are well known in the art, see, e.g., U.S. Pat. No. 5,830,696. For example, mutagens can be used to randomly mutate a gene. Mutagens include, e.g., ultraviolet light or gamma irradiation, or a chemical mutagen, e.g., mitomycin, nitrous acid, photoactivated psoralens, alone or in combination, to induce DNA breaks amenable to repair by recombination. Other chemical mutagens include, for example, sodium bisulfite, nitrous acid, hydroxylamine, hydrazine or formic acid. Other mutagens are analogues of nucleotide precursors, e.g., nitrosoguanidine, 5-bromouracil, 2-aminopurine, or acridine. These agents can be added to a PCR reaction in place of the nucleotide precursor thereby mutating the sequence. Intercalating agents such as proflavine, acriflavine, quinacrine and the like can also be used.

Any technique in molecular biology can be used, e.g., random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196. Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793. In alternative aspects, modifications, additions or deletions are introduced by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

The following publications describe a variety of recursive recombination procedures and/or methods which can be incorporated into the methods of the invention: Stemmer (1999) “Molecular breeding of viruses for targeting and other clinical properties” Tumor Targeting 4:1-4; Ness (1999) Nature Biotechnology 17:893-896; Chang (1999) “Evolution of a cytokine using DNA family shuffling” Nature Biotechnology 17:793-797; Minshull (1999) “Protein evolution by molecular breeding” Current Opinion in Chemical Biology 3:284-290; Christians (1999) “Directed evolution of thymidine kinase for AZT phosphorylation using DNA family shuffling” Nature Biotechnology 17:259-264; Crameri (1998) “DNA shuffling of a family of genes from diverse species accelerates directed evolution” Nature 391:288-291; Crameri (1997) “Molecular evolution of an arsenate detoxification pathway by DNA shuffling,” Nature Biotechnology 15:436-438; Zhang (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724733; Crameri et al. (1996) “Construction and evolution of antibody-phage libraries by DNA shuffling” Nature Medicine 2:100-103; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al. (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating variant sequences in practicing the methods of the invention include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional protocols for generating variant sequences in practicing the methods of the invention include point mismatch repair (Kramer (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

Additional protocols for generating variant sequences in practicing the methods of the invention include those discussed in U.S. Pat. Nos. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Additional protocols for generating variant sequences in practicing the methods of the invention are described in U.S. patent application Ser. No. (USSN) 09/407,800, “SHUFFLING OF CODON ALTERED GENES” by Patten et al. filed Sep. 28, 1999; “EVOLUTION OF WHOLE CELLS AND ORGANISMS BY RECURSIVE SEQUENCE RECOMBINATION” by del Cardayre et al., U.S. Pat. No. 6,379,964; “OLIGONUCLEOTIDE MEDIATED NUCLEIC ACID RECOMBINATION” by Crameri et al., U.S. Pat. Nos. 6,319,714; 6,368,861; 6,376,246; 6,423,542; 6,426,224 and PCT/US00/01203; “USE OF CODON-VARIED OLIGONUCLEOTIDE SYNTHESIS FOR SYNTHETIC SHUFFLING” by Welch et al., U.S. Pat. No. 6,436,675; “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g. “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); “METHODS OF POPULATING DATA STRUCTURES FOR USE IN EVOLUTIONARY SIMULATIONS” by Selifonov and Stemmer, filed Jan. 18, 2000 (PCT/US00/01138); and “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. Ser. No. 09/656,549); and U.S. Pat. Nos. 6,177,263; 6,153,410.

Non-stochastic, or “directed evolution,” methods

Exemplary protocols for generating variant sequences (e.g., modified nitroreductase-encoding sequences) in practicing the methods of the invention include non-stochastic, or “directed evolution,” methods, such as, e.g., saturation mutagenesis (GSSM), synthetic ligation reassembly (SLR), or a combination thereof. These methods can be used to modify the nucleic acids to generate nitroreductases with new or altered properties (e.g., enzyme activity under high or low acidic or alkaline conditions, high or low temperatures, high or low salt conditions and the like; different substrate affinity; enantioselective activity; modified antibody binding activity, etc.). Polypeptides encoded by the modified nucleic acids can be screened for an activity before testing for proteolytic or other activity. Any testing modality or protocol can be used, e.g., using a capillary array platform. See, e.g., U.S. Pat. Nos. 6,361,974; 6,280,926; 5,939,250.

Saturation mutagenesis, or, GSSM

In one aspect of the invention, non-stochastic gene modification, a “directed evolution process,” is used to generate nitroreductases with new or altered properties. Variations of this method have been termed “gene site-saturation mutagenesis,” “site-saturation mutagenesis,” “saturation mutagenesis” or simply “GSSM.” It can be used in combination with other mutagenization processes. See, e.g., U.S. Pat. Nos. 6,171,820; 6,238,884. In one aspect, GSSM comprises providing a template polynucleotide and a plurality of oligonucleotides, wherein each oligonucleotide comprises a sequence homologous to the template polynucleotide, thereby targeting a specific sequence of the template polynucleotide, and a sequence that is a variant of the homologous gene; generating progeny polynucleotides comprising non-stochastic sequence variations by replicating the template polynucleotide with the oligonucleotides, thereby generating polynucleotides comprising homologous gene sequence variations.

In one aspect, codon primers containing a degenerate N,N,G/T sequence are used to introduce point mutations into a polynucleotide, so as to generate a set of progeny polypeptides in which a full range of single amino acid substitutions is represented at each amino acid position, e.g., an amino acid residue in an enzyme active site or ligand binding site targeted to be modified. These oligonucleotides can comprise a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids. In one aspect, one such degenerate oligonucleotide (comprised of, e.g., one degenerate N,N,G/T cassette) is used for subjecting each original codon in a parental polynucleotide template to a full range of codon substitutions. In another aspect, at least two degenerate cassettes are used—either in the same oligonucleotide or not, for subjecting at least two original codons in a parental polynucleotide template to a full range of codon substitutions. For example, more than one N,N,G/T sequence can be contained in one oligonucleotide to introduce amino acid mutations at more than one site. This plurality of N,N,G/T sequences can be directly contiguous, or separated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

In one aspect, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g. in an oligonucleotide) a degenerate triplet sequence comprised of only one N, where said N can be in the first second or third position of the triplet. Any other bases including any combinations and permutations thereof can be used in the remaining two positions of the triplet. Alternatively, it may be desirable in some instances to use (e.g. in an oligo) a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). For example, for a 100 amino acid polypeptide, 2000 distinct species (i.e. 20 possible amino acids per position X 100 amino acid positions) can be generated. Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide molecules such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host, e.g., E. coli host, using, e.g., an expression vector) and subjected to expression screening. When an individual progeny polypeptide is identified by screening to display a favorable change in property (when compared to the parental polypeptide, such as increased proteolytic activity under alkaline or acidic conditions), it can be sequenced to identify the correspondingly favorable amino acid substitution contained therein.

In one aspect, upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid, and each of two favorable changes) and 3 positions. Thus, there are 3×3×3 or 27 total possibilities, including 7 that were previously examined—6 single point mutations (i.e. 2 at each of three positions) and no change at any position.

In another aspect, site-saturation mutagenesis can be used together with another stochastic or non-stochastic means to vary sequence, e.g., synthetic ligation reassembly (see below), shuffling, chimerization, recombination and other mutagenizing processes and mutagenizing agents. This invention provides for the use of any mutagenizing process(es), including saturation mutagenesis, in an iterative manner.

Synthetic Ligation Reassembly (SLR)

In practicing the methods of the invention a non-stochastic gene modification system termed “synthetic ligation reassembly,” or simply “SLR,” a “directed evolution process,” can be used to generate nitroreductases with new or altered properties. SLR is a method of ligating oligonucleotide fragments together non-stochastically. This method differs from stochastic oligonucleotide shuffling in that the nucleic acid building blocks are not shuffled, concatenated or chimerized randomly, but rather are assembled non-stochastically. See, e.g., U.S. Patent Application Ser. No. (USSN) 09/332,835 entitled “Synthetic Ligation Reassembly in Directed Evolution” and filed on Jun. 14, 1999 (“U.S. Ser. No. 09/332,835”). In one aspect, SLR comprises the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations.

SLR does not depend on the presence of high levels of homology between polynucleotides to be rearranged. Thus, this method can be used to non-stochastically generate libraries (or sets) of progeny molecules comprised of over 10100 different chimeras. SLR can be used to generate libraries comprised of over 101000 different progeny chimeras. Thus, aspects of the present invention include non-stochastic methods of producing a set of finalized chimeric nucleic acid molecule shaving an overall assembly order that is chosen by design. This method includes the steps of generating by design a plurality of specific nucleic acid building blocks having serviceable mutually compatible ligatable ends, and assembling these nucleic acid building blocks, such that a designed overall assembly order is achieved.

The mutually compatible ligatable ends of the nucleic acid building blocks to be assembled are considered to be “serviceable” for this type of ordered assembly if they enable the building blocks to be coupled in predetermined orders. Thus, the overall assembly order in which the nucleic acid building blocks can be coupled is specified by the design of the ligatable ends. If more than one assembly step is to be used, then the overall assembly order in which the nucleic acid building blocks can be coupled is also specified by the sequential order of the assembly step(s). In one aspect, the annealed building pieces are treated with an enzyme, such as a ligase (e.g. T4 DNA ligase), to achieve covalent bonding of the building pieces.

In one aspect, the design of the oligonucleotide building blocks is obtained by analyzing a set of progenitor nucleic acid sequence templates that serve as a basis for producing a progeny set of finalized chimeric polynucleotides. These parental oligonucleotide templates thus serve as a source of sequence information that aids in the design of the nucleic acid building blocks that are to be mutagenized, e.g., chimerized or shuffled. In one aspect of this method, the sequences of a plurality of parental nucleic acid templates are aligned in order to select one or more demarcation points. The demarcation points can be located at an area of homology, and are comprised of one or more nucleotides. These demarcation points are preferably shared by at least two of the progenitor templates. The demarcation points can thereby be used to delineate the boundaries of oligonucleotide building blocks to be generated in order to rearrange the parental polynucleotides. The demarcation points identified and selected in the progenitor molecules serve as potential chimerization points in the assembly of the final chimeric progeny molecules. A demarcation point can be an area of homology (comprised of at least one homologous nucleotide base) shared by at least two parental polynucleotide sequences. Alternatively, a demarcation point can be an area of homology that is shared by at least half of the parental polynucleotide sequences, or, it can be an area of homology that is shared by at least two thirds of the parental polynucleotide sequences. Even more preferably a serviceable demarcation points is an area of homology that is shared by at least three fourths of the parental polynucleotide sequences, or, it can be shared by at almost all of the parental polynucleotide sequences. In one aspect, a demarcation point is an area of homology that is shared by all of the parental polynucleotide sequences.

In one aspect, a ligation reassembly process is performed exhaustively in order to generate an exhaustive library of progeny chimeric polynucleotides. In other words, all possible ordered combinations of the nucleic acid building blocks are represented in the set of finalized chimeric nucleic acid molecules. At the same time, in another aspect, the assembly order (i.e. the order of assembly of each building block in the 5′ to 3 sequence of each finalized chimeric nucleic acid) in each combination is by design (or non-stochastic) as described above. Because of the non-stochastic nature of this invention, the possibility of unwanted side products is greatly reduced.

In another aspect, the ligation reassembly method is performed systematically. For example, the method is performed in order to generate a systematically compartmentalized library of progeny molecules, with compartments that can be screened systematically, e.g. one by one. In other words this invention provides that, through the selective and judicious use of specific nucleic acid building blocks, coupled with the selective and judicious use of sequentially stepped assembly reactions, a design can be achieved where specific sets of progeny products are made in each of several reaction vessels. This allows a systematic examination and screening procedure to be performed. Thus, these methods allow a potentially very large number of progeny molecules to be examined systematically in smaller groups. Because of its ability to perform chimerizations in a manner that is highly flexible yet exhaustive and systematic as well, particularly when there is a low level of homology among the progenitor molecules, these methods provide for the generation of a library (or set) comprised of a large number of progeny molecules. Because of the non-stochastic nature of the instant ligation reassembly invention, the progeny molecules generated preferably comprise a library of finalized chimeric nucleic acid molecules having an overall assembly order that is chosen by design. The saturation mutagenesis and optimized directed evolution methods also can be used to generate different progeny molecular species. It is appreciated that the invention provides freedom of choice and control regarding the selection of demarcation points, the size and number of the nucleic acid building blocks, and the size and design of the couplings. It is appreciated, furthermore, that the requirement for intermolecular homology is highly relaxed for the operability of this invention. In fact, demarcation points can even be chosen in areas of little or no intermolecular homology. For example, because of codon wobble, i.e. the degeneracy of codons, nucleotide substitutions can be introduced into nucleic acid building blocks without altering the amino acid originally encoded in the corresponding progenitor template.

Alternatively, a codon can be altered such that the coding for an originally amino acid is altered. This invention provides that such substitutions can be introduced into the nucleic acid building block in order to increase the incidence of intermolecular homologous demarcation points and thus to allow an increased number of couplings to be achieved among the building blocks, which in turn allows a greater number of progeny chimeric molecules to be generated.

In another aspect, the synthetic nature of the step in which the building blocks are generated allows the design and introduction of nucleotides (e.g., one or more nucleotides, which may be, for example, codons or introns or regulatory sequences) that can later be optionally removed in an in vitro process (e.g. by mutagenesis) or in an in vivo process (e.g. by utilizing the gene splicing ability of a host organism). It is appreciated that in many instances the introduction of these nucleotides may also be desirable for many other reasons in addition to the potential benefit of creating a serviceable demarcation point.

In one aspect, a nucleic acid building block is used to introduce an intron. Thus, functional introns are introduced into a man-made gene manufactured according to the methods described herein. The artificially introduced intron(s) can be functional in a host cells for gene splicing much in the way that naturally-occurring introns serve functionally in gene splicing.

Optimized Directed Evolution System

In practicing the methods of the invention a non-stochastic gene modification system termed “optimized directed evolution system” can be used to generate nitroreductases with new or altered properties. Optimized directed evolution is directed to the use of repeated cycles of reductive reassortment, recombination and selection that allow for the directed molecular evolution of nucleic acids through recombination. Optimized directed evolution allows generation of a large population of evolved chimeric sequences, wherein the generated population is significantly enriched for sequences that have a predetermined number of crossover events.

A crossover event is a point in a chimeric sequence where a shift in sequence occurs from one parental variant to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. This provides more control over choosing chimeric variants having a predetermined number of crossover events.

In addition, this method provides a convenient means for exploring a tremendous amount of the possible protein variant space in comparison to other systems. Previously, if one generated, for example, 10¹³ chimeric molecules during a reaction, it would be extremely difficult to test such a high number of chimeric variants for a particular activity. Moreover, a significant portion of the progeny population would have a very high number of crossover events which resulted in proteins that were less likely to have increased levels of a particular activity. By using these methods, the population of chimerics molecules can be enriched for those variants that have a particular number of crossover events. Thus, although one can still generate 10¹³ chimeric molecules during a reaction, each of the molecules chosen for further analysis' most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

One method for creating a chimeric progeny polynucleotide sequence is to create oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. Additional information can also be found, e.g., in U.S. Ser. No. 09/332,835; U.S. Pat. No. 6,361,974. The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PD F) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. By utilizing these methods, one can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events. These methods are directed to the use of repeated cycles of reductive reassortment, recombination and selection that allow for the directed molecular evolution of a nucleic acid encoding a polypeptide through recombination. This system allows generation of a large population of evolved chimeric sequences, wherein the generated population is significantly enriched for sequences that have a predetermined number of crossover events. A crossover event is a point in a chimeric sequence where a shift in sequence occurs from one parental variant to another parental variant. Such a point is normally at the juncture of where oligonucleotides from two parents are ligated together to form a single sequence. The method allows calculation of the correct concentrations of oligonucleotide sequences so that the final chimeric population of sequences is enriched for the chosen number of crossover events. This provides more control over choosing chimeric variants having a predetermined number of crossover events.

In addition, these methods provide a convenient means for exploring a tremendous amount of the possible protein variant space in comparison to other systems. By using the methods described herein, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

In one aspect, the method creates a chimeric progeny polynucleotide sequence by creating oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. See also U.S. Ser. No. 09/332,835.

The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. One can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events.

Iterative Processes

In practicing the invention, these processes can be iteratively repeated. For example a nucleic acid (or, the nucleic acid) responsible for an altered protein phenotype is identified, re-isolated, again modified, re-tested for activity using the methods of the invention. This process can be iteratively repeated until a desired phenotype is engineered. For example, an entire biochemical anabolic or catabolic pathway can be engineered into a cell, including proteolytic activity.

Similarly, if it is determined that a particular oligonucleotide has no affect at all on the desired trait (e.g., a nitroreductases phenotype), it can be removed as a variable by synthesizing larger parental oligonucleotides that include the sequence to be removed. Since incorporating the sequence within a larger sequence prevents any crossover events, there will no longer be any variation of this sequence in the progeny polynucleotides. This iterative practice of determining which oligonucleotides are most related to the desired trait, and which are unrelated, allows more efficient exploration all of the possible protein variants that might be provide a particular trait or activity.

Producing Sequence Variants

In practicing the methods of the invention nucleic acid variants can be generated using genetic engineering techniques such as site directed mutagenesis, random chemical mutagenesis, Exonuclease III deletion procedures, and standard cloning techniques. Alternatively, such variants, fragments, analogs, or derivatives may be created using chemical synthesis or modification procedures. Other methods of making variants are also familiar to those skilled in the art. These include procedures in which nucleic acid sequences obtained from natural isolates are modified to generate nucleic acids which encode polypeptides having characteristics which enhance their value in industrial or laboratory applications. In such procedures, a large number of variant sequences having one or more nucleotide differences with respect to the sequence obtained from the natural isolate are generated and characterized. These nucleotide differences can result in amino acid changes with respect to the polypeptides encoded by the nucleic acids from the natural isolates.

For example, variants may be created using error prone PCR. In error prone PCR, PCR is performed under conditions where the copying fidelity of the DNA polymerase is low, such that a high rate of point mutations is obtained along the entire length of the PCR product. Error prone PCR is described, e.g., in Leung, D. W., et al., Technique, 1:11-15, 1989) and Caldwell, R. C. & Joyce G. F., PCR Methods Applic., 2:28-33, 1992. Briefly, in such procedures, nucleic acids to be mutagenized are mixed with PCR primers, reaction buffer, MgCl₂, MnCl₂, Taq polymerase and an appropriate concentration of dNTPs for achieving a high rate of point mutation along the entire length of the PCR product. For example, the reaction may be performed using 20 fmoles of nucleic acid to be mutagenized, 30 pmole of each PCR primer, a reaction buffer comprising 50 mM KCl, 10 mM Tris HCl (pH 8.3) and 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR may be performed for 30 cycles of 94° C. for 1 min, 45° C. for 1 min, and 72° C. for 1 min. However, it will be appreciated that these parameters may be varied as appropriate. The mutagenized nucleic acids are cloned into an appropriate vector and the activities of the polypeptides encoded by the mutagenized nucleic acids is evaluated.

Variants may also be created using oligonucleotide directed mutagenesis to generate site-specific mutations in any cloned DNA of interest. Oligonucleotide mutagenesis is described, e.g., in Reidhaar-Olson (1988) Science 241:53-57. Briefly, in such procedures a plurality of double stranded oligonucleotides bearing one or more mutations to be introduced into the cloned DNA are synthesized and inserted into the cloned DNA to be mutagenized. Clones containing the mutagenized DNA are recovered and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, e.g., U.S. Pat. No. 5,965,408.

Still another method of generating variants is sexual PCR mutagenesis. In sexual PCR mutagenesis, forced homologous recombination occurs between DNA molecules of different but highly related DNA sequence in vitro, as a result of random fragmentation of the DNA molecule based on sequence homology, followed by fixation of the crossover by primer extension in a PCR reaction. Sexual PCR mutagenesis is described, e.g., in Stemmer (1994) Proc. Natl. Acad. Sci. USA 91:10747-10751. Briefly, in such procedures a plurality of nucleic acids to be recombined are digested with DNase to generate fragments having an average size of 50-200 nucleotides. Fragments of the desired average size are purified and resuspended in a PCR mixture. PCR is conducted under conditions which facilitate recombination between the nucleic acid fragments. For example, PCR may be performed by resuspending the purified fragments at a concentration of 10-30 ng/:1 in a solution of 0.2 mM of each dNTP, 2.2 mM MgCl₂, 50 mM KCL, 10 mM Tris HCl, pH 9.0, and 0.1% Triton X100. 2.5 units of Taq polymerase per 100:1 of reaction mixture is added and PCR is performed using the following regime: 94° C. for 60 seconds, 94° C. for 30 seconds, 50-55° C. for 30 seconds, 72° C. for 30 seconds (30-45 times) and 72° C. for 5 minutes. However, it will be appreciated that these parameters may be varied as appropriate. In some aspects, oligonucleotides may be included in the PCR reactions. In other aspects, the Klenow fragment of DNA polymerase I may be used in a first set of PCR reactions and Taq polymerase may be used in a subsequent set of PCR reactions. Recombinant sequences are isolated and the activities of the polypeptides they encode are assessed.

Variants may also be created by in vivo mutagenesis. In some aspects, random mutations in a sequence of interest are generated by propagating the sequence of interest in a bacterial strain, such as an E. coli strain, which carries mutations in one or more of the DNA repair pathways. Such “mutator” strains have a higher random mutation rate than that of a wild-type parent. Propagating the DNA in one of these strains will eventually generate random mutations within the DNA. Mutator strains suitable for use for in vivo mutagenesis are described, e.g., in PCT Publication No. WO 91/16427.

Variants may also be generated using cassette mutagenesis. In cassette mutagenesis a small region of a double stranded DNA molecule is replaced with a synthetic oligonucleotide “cassette” that differs from the native sequence. The oligonucleotide often contains completely and/or partially randomized native sequence. Recursive ensemble mutagenesis may also be used to generate variants.

Recursive ensemble mutagenesis is an algorithm for protein engineering (protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described, e.g., in Arkin (1992) Proc. Natl. Acad. Sci. USA 89:7811-7815.

In some aspects, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described, e.g., in Delegrave (1993) Biotechnology Res. 11:1548-1552. Random and site-directed mutagenesis are described, e.g., in Arnold (1993) Current Opinion in Biotechnology 4:450-455.

In some aspects, the variants are created using shuffling procedures wherein portions of a plurality of nucleic acids which encode distinct polypeptides are fused together to create chimeric nucleic acid sequences which encode chimeric polypeptides as described in, e.g., U.S. Pat. Nos. 5,965,408; 5,939,250.

Optimizing Codons to Achieve High Levels of Protein Expression in Host Cells

In one aspect of the invention, nucleic acids are mutated to modify codon usage. In one aspect, methods of the invention comprise modifying codons in a nucleic acid encoding a nitroreductase to increase or decrease its expression in a host cell, e.g., a bacterial, insect, mammalian, yeast or plant cell. The method can comprise identifying a “non-preferred” or a “less preferred” codon in protein-encoding nucleic acid and replacing one or more of these non-preferred or less preferred codons with a “preferred codon” encoding the same amino acid as the replaced codon and at least one non-preferred or less preferred codon in the nucleic acid has been replaced by a preferred codon encoding the same amino acid. A preferred codon is a codon over-represented in coding sequences in genes in the host cell and a non-preferred or less preferred codon is a codon under-represented in coding sequences in genes in the host cell.

Screening Methodologies and Devices

In practicing the methods of the invention, a variety of apparatus and methodologies can be used to determine the enzymatic activity of a nitroreductase, e.g., to screen for variant nitroreductases, including e.g., plates such as microtiter well plates and capillary arrays.

Capillary Arrays

Capillary arrays, such as the GIGAMATRIX™, Diversa Corporation, San Diego, Calif., can be used to practice the methods of the invention. Nucleic acids or polypeptides (nitroreductases) can be immobilized to or applied to an array, including capillary arrays. Arrays can be used to screen for the activity of a polypeptide, e.g., binding or enzymatic activity. Capillary arrays can provide another system for holding and screening samples. For example, a sample screening apparatus can include a plurality of capillaries formed into an array of adjacent capillaries, wherein each capillary comprises at least one wall defining a lumen for retaining a sample. The apparatus can further include interstitial material disposed between adjacent capillaries in the array, and one or more reference indicia formed within of the interstitial material. A capillary for screening a sample, wherein the capillary is adapted for being bound in an array of capillaries, can include a first wall defining a lumen for retaining the sample, and a second wall formed of a filtering material, for filtering excitation energy provided to the lumen to excite the sample.

A polypeptide can be introduced into a first component into at least a portion of a capillary of a capillary array. Each capillary of the capillary array can comprise at least one wall defining a lumen for retaining the first component. An air bubble can be introduced into the capillary behind the first component. A second component can be introduced into the capillary, wherein the second component is separated from the first component by the air bubble. A sample of interest can be introduced as a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method can further include removing the first liquid from the capillary tube, wherein the bound detectable particle is maintained within the capillary, and introducing a second liquid into the capillary tube.

The capillary array can include a plurality of individual capillaries comprising at least one outer wall defining a lumen. The outer wall of the capillary can be one or more walls fused together. Similarly, the wall can define a lumen that is cylindrical, square, hexagonal or any other geometric shape so long as the walls form a lumen for retention of a liquid or sample. The capillaries of the capillary array can be held together in close proximity to form a planar structure. The capillaries can be bound together, by being fused (e.g., where the capillaries are made of glass), glued, bonded, or clamped side-by-side. The capillary array can be formed of any number of individual capillaries, for example, a range from 100 to 4,000,000 capillaries. A capillary array can form a micro titer plate having about 100,000 or more individual capillaries bound together.

Arrays, or “Biochips”

In practicing the invention nucleic acids (e.g., nitroreductase-encoding) or polypeptides (e.g., nitroreductases) can be immobilized to or applied to an array. Arrays can be used to screen for or monitor libraries of compositions (e.g., antibodies, enzymes, etc.) for their ability to bind to or modulate the activity of a polypeptide. Polypeptide arrays” can be used to simultaneously quantify a plurality of proteins. The present invention can be practiced with any known “array,” also referred to as a “microarray” or “DNA array” or “nucleic acid array” or “polypeptide array” or “antibody array” or “biochip,” or variation thereof. Arrays are generically a plurality of “spots” or “target elements,” each target element comprising a defined amount of one or more biological molecules immobilized onto a defined area of a substrate surface for specific binding to a sample molecule. Any immobilization method can be used, e.g., immobilization upon an inert support such as diethylaminoethyl-cellulose, porous glass, chitin or cells. In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399407; Bowtell (1999) Nature Genetics Supp. 21:25-32. See also published U.S. patent applications Nos. 20010018642; 20010019827; 20010016322; 20010014449; 20010014448; 20010012537; 20010008765.

Antibodies and Antibody-Based Screening Methods

The methods of the invention can be used to generate modified nitroreductase-encoding nucleic acids and modified nitroreductases. Antibodies can be used to monitor the amount or the activity of a protein. The nitroreductase itself can be an antibody, i.e., a catalytic antibody with nitroreductase activity. Antibodies also can be used in immunoprecipitation, staining, immunoaffinity columns, and the like. Methods of doing assays, e.g., ELISAs, with polyclonal and monoclonal antibodies are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

The ability of proteins in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. Particular assays include ELISA assays, sandwich assays, radioimmunoassays, and Western Blots.

Kits

The invention provides kits comprising material for practicing the methods of the invention. The kits also can contain instructional material teaching the methodologies and industrial uses of the invention, as described herein.

A number of embodiments of the invention have been described. Nevertheless, it will be understood that various modifications may be made without departing from the spirit and scope of the invention. Accordingly, other embodiments are within the scope of the following claims.

What is claimed is:

1. A biocatalytic method for reducing a compound comprising a nitro group, the method comprising the following steps:
(a) providing a nitroreductase;
(b) providing a compound comprising a nitro group; and
(c) contacting the nitroreductase with the compound under conditions wherein the nitro group is reduced to an amine group.

2. The method of claim 1, wherein the contacting is performed in a reaction vessel.

3. The method of claim 1, wherein the contacting is performed in a cell extract.

4. The method of claim 1, wherein the contacting is performed in a whole cell.

5. The method of claim 4, wherein the cell is a bacterial cell, a yeast cell, a fungal cell, a plant cell, an insect cell or a mammalian cell.

6. The method of claim 1, wherein the nitroreductase is a recombinant, isolated or synthetically generated enzyme.

7. The method of claim 1, wherein the nitroreductase is a recombinant, isolated or synthetically generated biocatalytic antibody having a nitroreductase activity.

8. The method of claim 1, wherein the compound comprises a nitroaromatic compound and a substituted aniline is biocatalytically generated.

9. The method of claim 8, wherein the nitroaromatic compound is halosubstituted.

10. The method of claim 1, wherein the compound comprises a nitroalkyl group.

11. The method of claim 1, wherein the compound comprises a nitroalkane.

12. The method of claim 1, wherein the compound comprises a nitroaromatic.

13. The method of claim 12, wherein the nitroaromatic compound is 2-nitro-benzoic acid and the nitroreductase reduces 2-nitro-benzoic acid to anthranilic acid.

14. The method of claim 1, wherein the compound comprises a styrene.

15. The method of claim 1, wherein the compound comprises a racemic nitroalkane compound and a chiral amine is biocatalytically generated.

16. The method of claim 1, wherein the compound comprises a 4-nitrobuylamine or a 1,4-dinitro-butane and a putrescine is biocatalytically generated.

17. A method for selecting a nitroreductase that can catalyze the reduction of a nitro group to its corresponding amine comprising the following steps:
(a) providing a test sample comprising a polypeptide;
(b) providing a compound comprising a nitro group; and
(c) contacting the test sample with the compound comprising a nitro group and detecting the generation of a corresponding amine, wherein the generation of the corresponding amine indicates the presence of a nitroreductase in the test sample.

18. A method for selecting a nitroreductase that can catalyze the reduction of an aliphatic nitro group comprising the following steps:
(a) providing a test sample comprising a polypeptide;
(b) providing a 4-nitrobuylamine or a 1,4-dinitro-butane; and
(c) contacting the test sample with the 4-nitrobuylamine or the 1,4-dinitrobutane and detecting the generation of putrescine, wherein the generation of putrescine indicates the presence of a nitroreductase in the test sample.

19. The method for selecting a nitroreductase that can catalyze the reduction of an aliphatic nitro group comprising the following steps:
(a) providing a test sample comprising a polypeptide;
(b) providing a 4-nitrobuylamine or a 1,4-dinitro-butane; and
(c) contacting the test sample with the 4-nitrobuylamine or the 1,4-dinitrobutane in an in vivo system or equivalent system and detecting the generation of succinate, wherein the generation of succinate indicates the presence of a nitroreductase in the test sample.

20. The method of claim 18 or claim 19, wherein the putrescine or the succinate is detected by on-line HPLC or by using a mass spectograph.

21. A method for selecting a nitroreductase that can catalyze the reduction of an aliphatic nitro group comprising the following steps:
(a) providing a test sample comprising a polypeptide;
(b) providing a 2-nitro-benzoic acid; and
(c) contacting the test sample with the 2-nitro-benzoic acid system and detecting the generation of anthranilic acid, wherein the generation of anthranilic acid indicates the presence of a nitroreductase in the test sample.

22. The method of claim 20 or claim 21, wherein the anthranilic acid is detected by fluorescence, on-line HPLC or by using a mass spectograph.

23. A method for selecting a nitroreductase that can catalyze the reduction of an aliphatic nitro group comprising the following steps:
(a) providing a test sample comprising a polypeptide;
(b) providing a 2-nitro-benzoic acid; and
(c) contacting the test sample with the 2-nitro-benzoic acid and detecting the generation of anthranilic acid, wherein the generation of anthranilic acid indicates the presence of a nitroreductase in the test sample.

24. A method for selecting a nitroreductase that can catalyze the reduction of an aliphatic nitro group comprising the following steps:
(a) providing a test sample comprising a polypeptide;
(b) providing a 2-nitro-benzoic acid; and
(c) contacting the test sample with the 2-nitro-benzoic acid in an in vivo system or equivalent system and detecting the generation of tryptophan, wherein the generation of tryptophan indicates the presence of a nitroreductase in the test sample.

25. A method for selecting a nitroreductase that can catalyze the reduction of an aliphatic nitro group comprising the following steps:
(a) providing a test sample comprising a polypeptide;
(b) providing a nitroacid; and
(c) contacting the test sample with the nitroacid and detecting the generation of an amino acid, wherein the generation of the amino acid indicates the presence of a nitroreductase in the test sample.

26. The method of claim 25, wherein the test sample and the nitroacid are contacted in an in vivo system.

27. The method of claim 26, wherein the in vivo system is an amino acid auxotroph.

28. A method for selecting a nitroreductase that can catalyze the reduction of a halogenated nitroaromatic compound comprising the following steps:
(a) providing a test sample comprising a polypeptide;
(b) providing a halogenated nitroaromatic compound; and
(c) contacting the test sample with the halogenated nitroaromatic compound and detecting the generation of a corresponding amine, wherein the generation of the corresponding amine indicates the presence of a nitroreductase in the test sample.

29. A method for selecting a nitroreductase that can catalyze the reduction of a styrene comprising the following steps:
(a) providing a test sample comprising a polypeptide;
(b) providing a styrene; and
(c) contacting the test sample with the styrene and detecting the generation of a corresponding amine, wherein the generation of the corresponding amine indicates the presence of a nitroreductase in the test sample.

30. A method for selecting a nitroreductase that can catalyze the reduction of an aliphatic nitrocompound comprising the following steps:
(a) providing a test sample comprising a polypeptide;
(b) providing an aliphatic nitrocompound; and
(c) contacting the test sample with the aliphatic nitrocompound and detecting the generation of a corresponding amine, wherein the generation of the corresponding amine indicates the presence of a nitroreductase in the test sample.

31. The method of claim 30, wherein the corresponding amine is a chiral amine.

32. A method for selecting a nitroreductase that can catalyze the reduction of an asymmetric nitroaldol comprising the following steps:
(a) providing a test sample comprising a polypeptide;
(b) providing an asymmetric nitroaldol;
(c) contacting the test sample with the asymmetric nitroaldol and detecting the generation of a corresponding amine, wherein the generation of the corresponding amine indicates the presence of a nitroreductase in the test sample.

33. A method for chemoselective reduction of a nitro group, the method comprising the following steps:
(a) providing a nitroreductase;
(b) providing a nitro group; and
(c) contacting the nitroreductase with the nitro group under conditions wherein the nitroreductase catalyzes the reduction of the nitro group to an amine group.

34. A biocatalytic method for making a substituted aniline, the method comprising the following steps:
(a) providing a nitroreductase;
(b) providing a nitroaromatic compound; and
(c) contacting the nitroreductase with the nitroaromatic compound under conditions wherein the nitroreductase catalyzes the reduction of the nitro group to an amine group and a substituted aniline is generated.

35. The method of claim 34, wherein the nitroaromatic compound comprises a halogenated nitroaromatic compound.

36. A biocatalytic method for reducing a styrene, the method comprising the following steps:
(a) providing a nitroreductase;
(b) providing a styrene; and
(c) contacting the nitroreductase with the styrene under conditions wherein the nitroreductase catalyzes the reduction of the nitro group to an amine group and the styrene is reduced to its corresponding amine.

37. A biocatalytic method for making a chiral amine, the method comprising the following steps:
(a) providing a nitroreductase;
(b) providing a racemic nitroalkane compound; and
(c) contacting the nitroreductase with the nitroalkane under conditions wherein the nitroreductases reduces the nitroalkane to generate a chiral amine.

38. The method of claim 37, wherein the contacting takes place at a neutral pH such that a dynamic kinetic resolution is effected.

39. The method of claim 37, wherein the nitroalkane compound comprises a benzylic nitro group.

40. A biocatalytic method for making putrescine, the method comprising the following steps:
(a) providing a nitroreductase;
(b) providing a 4-nitrobuylamine or a 1,4-dinitro-butane; and
(c) contacting the nitroreductase with the 4-nitrobuylamine or 1,4-dinitrobutane under conditions wherein the nitroreductases reduces the 4-nitrobuylamine or 1,4-dinitro-butane to generate a putrescine.

41. A method for selecting a nucleic acid encoding a nitroreductase, the method comprising the following steps:
(a) providing a plurality of nucleic acids;
(b) expressing the nucleic acids in a system comprising a nitro compound; and
(c) detecting the system where the nitro compound has been reduced to its corresponding amine, thereby selecting a nucleic acid encoding a nitroreductase.

42. The method of claim 41, wherein the plurality of nucleic acids comprises a genomic library or a cDNA library.

43. The method of claim 42, wherein the plurality of nucleic acids comprises an environmental library.

44. The method of claim 41, wherein the system lacks or has insubstantial amounts of an endogenous nitroreductase.

45. The method of claim 41, wherein the plurality of nucleic acids are cloned in an expression cassette.

46. The method of claim 45, wherein the expression cassette comprises a phage, a phagemid, a plasmid or a recombinant virus.

47. The method of claim 41, wherein the system comprises an in vitro system.

48. The method of claim 41, wherein the system comprises a cell.

49. The method of claim 48, wherein the cell is a bacterial cell.

50. The method of claim 48, wherein the corresponding amine is a cell growth factor or a compound the cell lacks in sufficient amount that is necessary for cell growth or survival, and expression of a nitroreductase reduces the nitro compound to its corresponding amine such that only cells expressing a nitroreductase proliferate or survive, thereby allowing selection of a nucleic acid encoding a nitroreductase.

51. The method of claim 50, wherein the nitro compound comprises a 4-nitrobuylamine or a 1,4-dinitro-butane and the corresponding amine growth factor is putrescine.

52. The method of claim 48, wherein the corresponding amine is a precursor or an intermediate in the synthesis of a cell growth factor or a compound that is necessary for cell growth or survival that the cell lacks in sufficient amounts, and expression of a nitroreductase reduces the nitro compound to its corresponding amine such that only cells expressing a nitroreductase proliferate or survive, thereby allowing selection of a nucleic acid encoding a nitroreductase.

53. The method of claim 52, wherein the nitro compound comprises a 4-nitrobuylamine or a 1,4-dinitro-butane and the corresponding amine that the cell lacks in sufficient amount that is necessary for cell growth is succinate, expression of a nitroreductase generates sufficient succinate for the cell to proliferate or survive.

54. The method of claim 52, wherein the cell lacks or substantially lacks anthranilate synthase activity and the ability to generate tryptophan, the nitro compound comprises a 2-nitro-benzoic acid, and expression of a nitroreductase generates sufficient tryptophan for the cell to proliferate or survive.

55. The method of claim 52, wherein the nitro compound comprises a nitro acid and the corresponding amine that the cell lacks in sufficient amount that is necessary for cell growth is an amino acid, and expression of a nitroreductase generates sufficient amino acid for the cell to proliferate or survive.

56. The method of claim 55, wherein the cell is an amino acid auxotroph.

57. The method of claim 56, wherein the cell is a leucine auxotroph, a proline auxotroph or a tryptophan auxotroph.

58. A method for making a nucleic acid encoding a nitroreductase capable of catalyzing reduction of a nitro compound in a set of conditions, the method comprising the following steps:
(a) providing a nucleic acid encoding a first nitroreductase;
(b) modifying the nitroreductase-encoding nucleic acid to generate a plurality of modified nucleic acids;
(c) expressing the modified nucleic acids in a system comprising a nitro compound in the set of conditions and detecting if the nitro compound has been reduced to its corresponding amine; and
(d) detecting which modified nucleic acid encoded the nitroreductases that catalyzed the reduction of the nitro compound to its corresponding amine, thereby making a nucleic acid encoding a modified nitroreductase capable of catalyzing reduction of a nitro compound in a set of conditions.

59. A method for making a nitroreductase capable of catalyzing reduction of a nitro compound in a set of conditions, the method comprising the following steps:
(a) providing a nucleic acid encoding a first nitroreductase;
(b) modifying the nitroreductase-encoding nucleic acid to generate a plurality of modified nucleic acids;
(c) expressing the modified nucleic acids in a system comprising a nitro compound in the set of conditions and detecting if the nitro compound has been reduced to its corresponding amine; and
(d) detecting which nitroreductase catalyzed the reduction of the nitro compound to its corresponding amine, thereby making a modified nitroreductase capable of catalyzing reduction of a nitro compound in a set of conditions.

60. The method of claim 58 or 59, wherein the first nitroreductase is not active in the selected set of conditions.

61. The method of claim 58 or 59, wherein the conditions in which the modified nitroreductase has optimal activity differs from the conditions in which the first nitroreductase has optimal activity.

62. The method of claim 58 or 59, wherein the modified nitroreductase acts on a different substrate than the first nitroreductase.

63. The method of claim 58 or 59, wherein the modified nitroreductase produces a different product than the first nitroreductase.

64. The method of claim 58 or 59, wherein the modified nitroreductases has a different co-factor requirement that the first nitroreductase.

65. The method of claim 58 or 59, wherein the set of conditions comprise high or low temperatures, high or low pH or high or low salt conditions.

66. The method of claim 58 or 59, wherein the nucleic acids expressing the modified nitroreductases are cloned in expression vehicles.

67. The method of claim 66, wherein the expression vehicle is a phage.

68. The method of claim 66, wherein the expression vehicle is an expression vector.

69. The method of claim 58 or 59, wherein the modified nucleic acids or modified nitroreductases are expressed in a well in a microtiter plate.

70. The method of claim 58 or 59, wherein the modified nucleic acids or modified nitroreductases are expressed in a capillary tube.

71. The method of claim 70, wherein the capillary comprises a capillary array.

72. The method of claim 71, wherein the capillary array comprises GIGAMATRIX™.

73. The method of claim 58 or 59, wherein a modified nucleic acid sequence is generated by a method comprising gene site saturated mutagenesis (GSSM).

74. The method of claim 58 or 59, wherein a modified nucleic acid sequence is generated by a method selected from the group consisting of gene site saturated mutagenesis (GSSM), error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, synthetic ligation reassembly (SLR) and a combination thereof.

75. The method of claim 58 or 59, wherein a modified nucleic acid sequence is generated by a method selected from the group consisting of recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation and a combination thereof.

76. The method of claim 58 or 59, wherein method is repeated iteratively to generate a modified nitroreductase having a desired activity under a particular set of conditions, a modified nitroreductase using a desired substrate or a modified nitroreductase generating a desired product.
